(19) United States
(12) Patent Application Publication
(10) Pub. No.: US 2017/0095231 A1
(43) Pub. Date: Apr. 6, 2017

(54) PORTABLE MEDICAL ULTRASOUND SCANNING SYSTEM HAVING A VIRTUAL USER INTERFACE

(71) Applicant: Sonoscanner SARL, Paris (FR)

(72) Inventors: Bruno Richard, Paris (FR); Etienne Richard, Paris (FR); Pierre-Adrien Nadal, Paris (FR)

(21) Appl. No.: 14/872,616

(22) Filed: Oct. 1, 2015

(51) Int. Cl. A61B 8/00

(52) U.S. Cl. CPC A61B 8/467; A61B 8/4427; A61B 8/465; A61B 8/54

(57) ABSTRACT

A portable ultrasound scanning system for performing a diagnostic ultrasound scanning process of a patient, comprising a portable housing configured to be held in a first hand of a user of the scanning system. A display comprising a touchscreen is mounted on the hand-held housing. A user input interface, at least a part of which is presented on the display, is configured to receive inputs from a user via the touchscreen and to transmit those inputs to the ultrasound electronics. A scanning probe is configured to be held in the user's second hand. A virtual control area is included in the user interface, the virtual control area being displayed on the touchscreen of the portable housing. A trackball zone is included in the virtual control area, the trackball zone performing a navigational function using a touch-sensitive manipulation method.

### FIELD OF THE DISCLOSURE

The present disclosure generally relates to portable medical ultrasound scanning systems, and in particular relates to a portable medical ultrasound scanning system having a virtual user interface.

### BACKGROUND OF THE INVENTION

Conventional medical scanners, such as ultrasound scanning systems, provide two- or three-dimensional images of tissues, including internal organs, without subjecting patients to ionizing radiation. These conventional scanners typically comprise a control system, a user interface (e.g., a keyboard, control panel, trackball, touchpad and the like), at least one probe (e.g., transducers or scanheads), a video display, a recording device, and a power system. Generally, ultrasound refers to sound waves emitted at frequencies above the range of human hearing. For diagnostic imaging, frequencies ranging from 2 to 15 megahertz are typically used.

Most conventional full-sized systems (desk or cart mounted) have the video display and the user interface directly attached or connected to each other for facilitating an intimate examination of the scanned images by an operator, such as an ultrasound technician or a physician.

Typically, the user interface, such as a trackball, mouse or touchpad, is used for performing specific functions during the diagnostic imaging process. For example, the trackball or touchpad manipulates color box positions and pulse wave window positions of a scanned structure or object, and measures its size using a digital caliper.

There have also been provided relatively small, portable ultrasound scanning systems (laptop computer sized) or ultraportable scanning systems (tablet computer sized) in which at least a part of the user interface is located on or close to the display screen, however, the use of such interfaces by the medical personnel operating the ultrasound scanning system is difficult and cumbersome since the user has one hand occupied with holding and manipulating the probe against the patient's body and the other hand is occupied in holding the remainder of the scanning system (the scanner housing). In those situations where a small handheld housing unit is provided with a touchscreen, moving a pointer or cursor on the touchscreen becomes difficult or imprecise in that the user's finger interferes with a zone of interest shown on the small display of the handheld unit.

Therefore, there is a need for developing a portable medical scanning system having an improved user interface and one that is readily operable with high precision during the diagnostic imaging process.

### SUMMARY OF THE INVENTION

Advantages are achieved by the present portable medical scanning system which includes a virtual user interface. An important aspect of the present medical scanning system is that a virtual control area is used to access a large number of functions, such as localizing a pulse wave (PW) gate, localizing a region or zone of interest (ROI) in various color modes, and performing accurate measurements of a scanned structure or object.

For example, the virtual control area is displayed on a touchscreen ultrasound device, and includes a simulated trackball zone for performing one or more mouse or trackball navigational functions. Also, one or more virtual buttons are displayed on the touchscreen for controlling various imaging system functions, such as manipulation of locations of display elements such as the pulsed-wave (PW) gate, the region of interest (ROI), and digital calipers. Instead of using a conventional physical knob or rolling member, the virtual control area is used to activate the imaging system functions.

In the present medical scanning system, a user has access to all necessary functions of the virtual control area using only one finger, such as a thumb, of the hand holding the system housing, without interfering with the region or zone of interest. For example, the virtual control area is displayed on a screen of the ultrasound device, near an edge of the device, such as in a lower left corner of the screen. It is contemplated that the virtual control area can be displayed in any corner or along any peripheral edge of the screen to suit different applications.

In one embodiment, a portable ultrasound scanning system is provided for performing a diagnostic scan of a patient, and includes a portable housing configured to be held in a first hand of a user of the scanning system with ultrasound electronics of the ultrasound scanning system contained within the hand-held housing and the housing having a touchscreen display. A user interface is installed in the portable housing, at least a part of which is presented on the display, and being configured to receive inputs from the user via the touchscreen and to transmit those inputs to the ultrasound electronics. A scanning probe is arranged to be in communication with the ultrasound electronics and is configured to be held in a second hand of the user of the scanning system. A virtual control area is included in the virtual user interface, and the virtual control area is displayed on the touchscreen of the portable housing. A trackball zone of the virtual control area performs a navigational function using a touch-sensitive manipulation method.

In another embodiment, at least a portion of the trackball zone is displayed at least one of: in one corner and along a peripheral edge of the touchscreen.

In another embodiment, at least a portion of the trackball zone is displayed on the touchscreen in an area accessible by at least one finger of the first hand of the user that is holding the portable housing.

In another embodiment, the entire trackball zone is accessible by at least one finger of the first and of the user that is holding the portable housing.

In another embodiment, the at least one finger comprises the thumb of the user.

In another embodiment, the virtual control area includes a virtual button arc disposed around a portion of the trackball zone.

In another embodiment, the trackball zone comprises a shape having an arcuate edge portion.

In an embodiment, the virtual arc is accessible by the at least one finger of the first hand of the user that is holding the portable housing.

In another embodiment, the virtual button arc has a plurality of virtual buttons displayed on the touchscreen for controlling at least one imaging system function.

In another embodiment, the virtual buttons are each associated with different imaging system functions.

In another embodiment, the virtual buttons are each associated with a single imaging system function.

In another embodiment, a touching of a first of the virtual buttons causes a display of different legends on the remaining virtual buttons based on an imaging system function associated with the first of the virtual buttons.

The foregoing and other aspects and features of the disclosure will become apparent to those of reasonable skill in the art from the following detailed description, as considered in conjunction with the accompanying drawings.

### BRIEF DESCRIPTION OF THE DRAWINGS

FIGS. 1A-1B illustrate an exemplary environment for the present medical scanning system in accordance with an embodiment of the present disclosure, utilizing a portable housing with a display screen and a connected probe;

FIG. 2 illustrates an exemplary practice of use of the present medical scanning system of FIG. 1 during the ultrasound scanning of a patient;

FIGS. 3-5 illustrate an exemplary display screen of the portable hosing of FIG. 1, featuring the user interface and the virtual display area and images associated with the scanned patient;

FIGS. 6A-6C illustrate various exemplary buttons of the user interface of FIGS. 3-5; and

FIG. 7 illustrates an exemplary display screen of the portable housing of FIG. 1, transitioning the virtual user interface into another configuration.

### DETAILED DESCRIPTION OF THE PREFERRED EMBODIMENTS

Preferred embodiments of the present disclosure are described below by way of example only, with reference to the accompanying drawings. Further, the following description is merely exemplary in nature and is in no way intended to limit the disclosure, its application, or uses.

As used herein, the term “module,” or “unit” may refer to, be part of, or include a programmable integrated circuit, an electronic circuit, a processor (shared, dedicated, or group) and/or memory (shared, dedicated, or group) that execute one or more software or firmware programs, a combinational logic circuit, and/or other suitable components that provide the described functionality.

Although children modules residing in their respective parent modules are shown, the broad teachings of the present system can be implemented in a variety of forms. Thus, while this disclosure includes particular examples and arrangements of the modules, the scope of the present system should not be so limited since other modifications will become apparent to the skilled practitioner.

Exemplary embodiments herein below are directed primarily to medical ultrasound scanning systems. However, the present system can be implemented for other types of ultrasound products or services. Thus, the terms “patient” and “tissue” are used to refer to the objects being scanned, and should be understood to refer also to other types of objects.

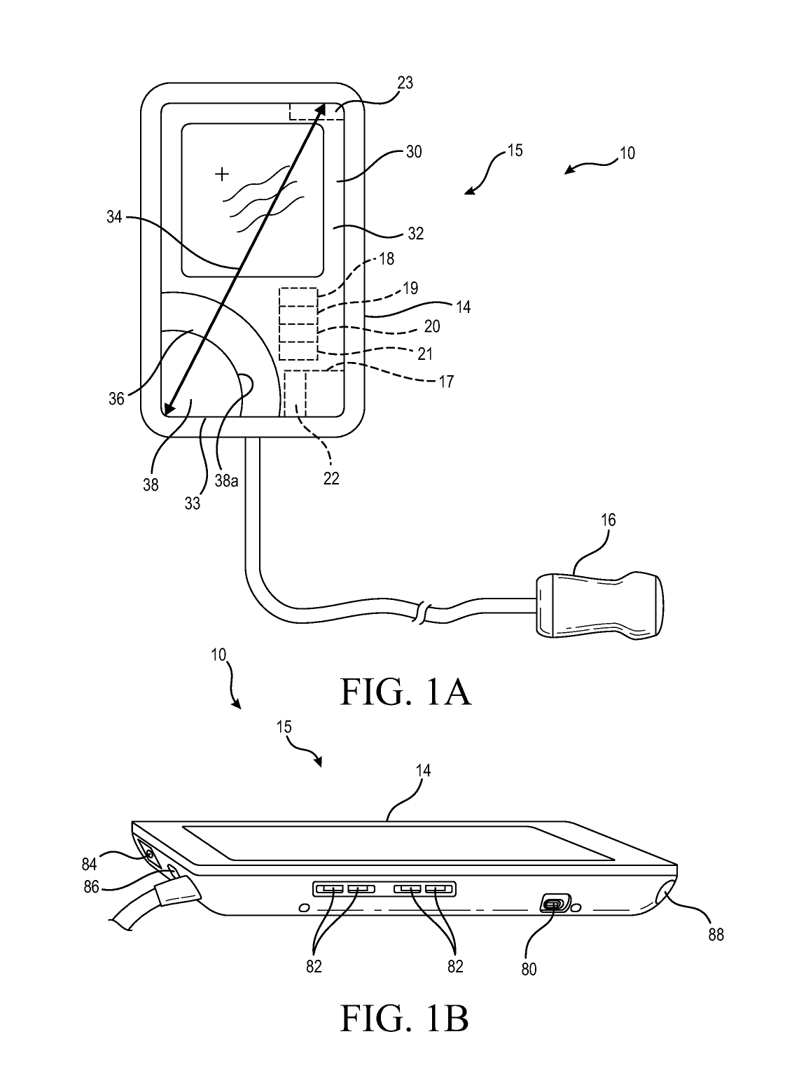

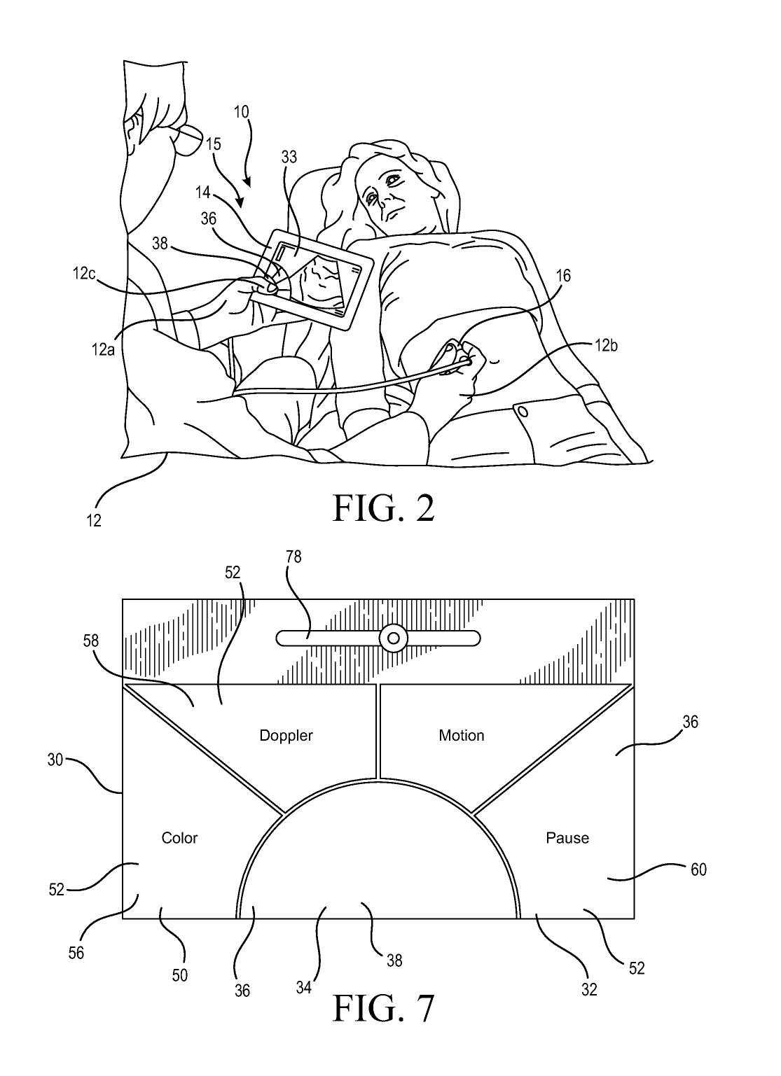

Referring now to FIGS. 1A, 1B and 2, an exemplary medical ultrasound scanning system for performing a diagnostic imaging process is schematically illustrated and generally designated 10. The present system 10 enables an operator 12 to perform the diagnostic ultrasound imaging process using a portable hand-held housing 14 and a probe 16. The housing 14 is preferably sized to be carried in one hand of an adult, such as a first hand 12a of the user 12, and preferably does not exceed a size of 30 cm by 30 cm (12 inches by 12 inches) with a thickness not exceeding 5 cm (2 inches). The weight of the housing 14, with all internal components discussed below, preferably does not exceed 1 kilogram (2.2 pounds). In a preferred embodiment, the hand-held housing 14 has a size of approximately 12.7 cm (5 inches) by 17.8 cm (7 inches), with a thickness of about 2 cm (0.9 inches), which allows the housing to fit and easily be carried in a standard size pocket of a medical personnel lab coat. The hand-held housing 14 preferably has a weight of about 600 gm (1.3 pounds) or less. Other sized housings 14 may be used with the present invention, but in a preferred embodiment, the housing has a weight of less than 1000 gm (2.2 pounds), more preferably a weight of less than 800 gm (1.8 pounds) and most preferably, a weight of no more than 600 gm (1.3 pounds).

The hand-held housing 14 contains ultrasound electronics 15 of an ultrasound scanner. Different embodiments may contain different components of the ultrasound electronics within the hand-held housing 14, with some embodiments containing all of the components, except, of course, the piezoelectric transducers of the probe. The ultrasound electronics include a beamformer 17, a central processing unit with a control module 18, a computing unit 19, a memory unit 20, an image processing unit 21, a power supply 22, and an optional wireless transmitter and receiver 23, all electrically interconnected. The ultrasound electronics 15 are configured to contain and operate conventional hardware, firmware and/or software for operating an ultrasound scanner in a known manner and displaying the scanned image on a display, as discussed below. The power supply 22 preferably comprises a rechargeable battery, such as a lithium polymer battery capable of providing several hours of operation for the ultrasound scanning system 10. Other types of power supplies including line power (alternating current) or other types of power storage devices, including different battery types, may be used as well. It is contemplated that the line power may be used in conjunction with a power converter, such as an AC-DC adapter.

A first display 30, comprising a touchscreen 32, is mounted on the hand-held housing 14. A user input interface 33 is provided in the housing 14, at least a part of which is presented on the first display 30. The user input interface 33 is configured to receive inputs from the user via the touchscreen 32 and to transmit those inputs to the central processing unit 18. Preferably the display 30 has a display area 34 of a size in the range of 10.2 cm (4 inches) to 38.1 cm (15 inches) as measured on the diagonal of the rectangular display area. In a preferred embodiment, the display 30 has a display area 34 of a size of about 17.8 cm (7 inches) as measured on the diagonal.

In some embodiments, a second or additional displays may be connected to the ultrasound electronics to provide for a larger display of the scanned image and to provide for a larger area for the user input interface 33 on the touchscreen 32 of the housing. A particular arrangement for an additional display is described in more detail in co-pending U.S. patent application Ser. No. 14/______ (attorney docket number 5490.120265), the disclosures of which are incorporated herein by reference.

The ultrasound electronics 15 are coupled to the probe 16 to direct controlling signals to the probe and to receive transducer signals from the probe.

More specifically, the probe 16 can be either directly connected or wirelessly connected to the ultrasound electronics 15. A particular type of detaching and reattaching wired arrangement for the probe 16, and particular types of probes that may be utilized, are disclosed and described in co-pending U.S. patent application Ser. No. 14/______ (attorney document number 5940.120267), the disclosures of which are incorporated herein by reference.

All relevant information can be stored in the memory unit 20, e.g., as a data storage device and/or a machine readable data storage medium carrying computer programs, for retrieval by the central processing unit 18 and its children modules as desired. For example, the central processing unit 18 may include an interface module for providing an interface between the central processing unit 18, the memory unit 20, and the display 30. The interface module controls operation of, for example, various components of the ultrasound electronics 15, the probe 16, the display 30, and other related system devices, services, and applications. The other devices, services, and applications may include, but are not limited to, one or more software or hardware components, etc. The interface module also receives signals, which are communicated to the respective modules, such as the central processing unit 18, and its children modules.

It is preferred that the ultrasound electronics 15 further includes a high definition multimedia interface (HDMI) port 80 for providing a multimedia connection with another electronic device, and at least one air vent outlet 82 for cooling the present system 10. Other auxiliary electronics include, but are not limited to, a power supply socket 84 for electrically charging the present system 10, at least one Universal Serial Bus (USB) port 86, and a power button 88 for turning on and off the present system.

Referring now to FIG. 2, an exemplary practice of the present medical ultrasound scanning system 10 is illustrated, utilizing the portable housing 14 having the user interface 33, and the probe 16. For example, the operator 12 holds the housing 14 having the user interface 33 with the first hand 12a, and manipulates the probe 16 against the skin or in a body cavity of a patient with a second hand 12b.

In a preferred embodiment, the user interface 33 includes a virtual control area 36 being displayed on the touchscreen 32 of the portable housing 14, and further includes a simulated or virtual trackball zone 38 for performing mouse or trackball navigational functions, using a touch-sensitive manipulation method as known in the art.

Referring now to FIGS. 2-5, it is contemplated that at least a portion of the trackball zone 38 is displayed in a lower left corner of the touchscreen 32, such that when a finger, such as a thumb 12c, of the first hand 12a of the operator 12 touches a surface of the touchscreen, the trackball zone can be virtually rotated in any desired direction for moving a cursor or a selected display element on the touchscreen, as discussed below. An outer edge 38a of the trackball zone 38 has a curved shape, thus giving the trackball zone the shape of one quarter of a circle when the trackball zone is located in a corner of the touchscreen 32, and thus the appearance of part of a familiar trackball. It is also contemplated that the virtual control area 36 can be displayed in any corner or along a peripheral edge of the touchscreen 32 to suit different applications, so long as the virtual control area is accessible by at least one finger 12c of the first hand 12a of the operator 12, while the housing 14 is being held in that first hand. The ultrasound electronics 15 may include sensors to detect an orientation of the housing 14 as it is held, so as to change the position of the virtual control area 36 as the housing is rotated by the user, such as between a landscape position and a portrait position of the display 30 of the housing 14. Other two-dimensional pointing device areas, in lieu of the virtual trackball zone 38, are also alternatively contemplated for use with the system to suit different applications.

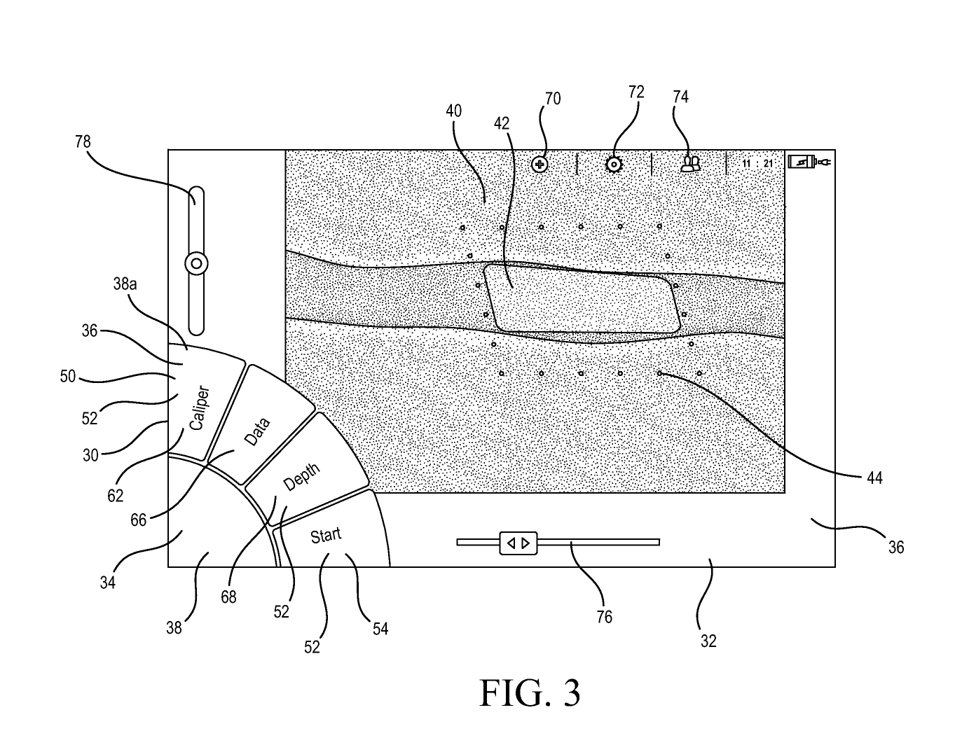

In a certain mode of operation, when the finger of the operator 12 moves on the trackball zone 38, such as by sliding or swiping, a corresponding ROI window 44 (selected display element) changes its location or position relative to the image of the scanned tissue of the patient 42, based on a positional information of the trackball zone. The ROI window 44 is defined by any demarcation method known in the art, such as a dotted line shown in FIG. 3.

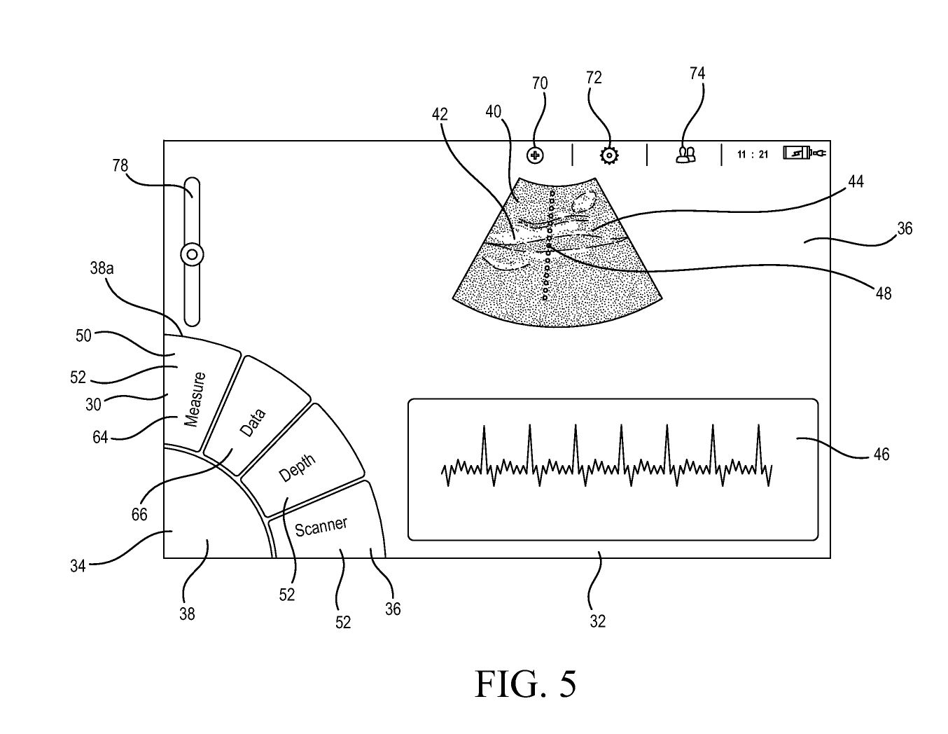

It is preferred that, as illustrated in FIG. 5, a signal window 46 is provided in a lower portion of the touchscreen 32 for viewing the PW Doppler signals associated with the scanned patient 42.

As shown in FIG. 5, it is also contemplated that a PW gate or cursor 48 is generated and localized (or focused) on an image 40 of the scanned patient 42 by selectively pressing on a desired portion of the image for a predetermined time period, or by rolling the trackball zone to move the PW gate to a desired position and “selecting” that position. The PW gate or cursor 48 generally refers to a location where the PW Doppler signal is listened to in the image 40. As similarly with the ROI window 44, when the finger of the operator 12 moves on the trackball zone 38, a corresponding location of the PW gate or cursor 48 changes based on the positional information of the trackball zone. As a result, the PW gate or cursor 48 precisely changes its relative position and size based on the movement of the trackball zone 38, such that a fingertip of the operator 12 does not mask the zone of interest on the image of the scanned patient 42. Similarly, the relative position and size of the ROI window 44 can be adjusted or manipulated by moving the finger of the operator 12 on the trackball zone 38.

Referring now to FIGS. 3-5, the virtual control area 36 includes a virtual button arc 50 disposed around the trackball zone 38 wherein the arc has at least one virtual button 52 being displayed on the touchscreen 32 for controlling various imaging system functions. It is contemplated that the arc 50 can be readily activated by the finger 12c of the first hand 12a of the operator 12 while that hand is holding the housing 14. Although the arc 50 is shown as having a partial circular shape, other suitable geometric shapes, such as linear and curvy shapes, are also contemplated to suit different applications.

An exemplary imaging system function of each virtual button 52 may include, but is not limited to, controlling a digital caliper, viewing associated data of the scanned patient 42, measuring a depth of the tissue being scanned, performing a start/stop operation of the present system 10, or the like. An important aspect of at least some of the virtual buttons 52 is that when the button is touched, other virtual buttons on the arc 50 change their designations such that the buttons are context sensitive and thus display legends and provide associated functions and their sub-functions, when touched, appropriate to the state of the scanner system 10 at any given time. The current state of the scanner system 10 may refer to, for example, a live scanning mode and a pause mode.

Referring now to FIGS. 3-5, 6A-6C, and 7, and merely as an example of one of limitless operating arrangements and displays, when a Start button 54 (FIG. 3) is activated, the present system 10 transitions into the live scanning mode. During the live scanning mode, a Color button 56 (FIG. 7) may be depressed by the finger of the operator 12 for a predetermined time period to activate and display buttons for the corresponding functions and sub-functions relating to the state of the scanner system 10 relating to the Color selection, such as steering left, steering right, and no steering of the image 40. A Doppler button 58 (FIG. 7) operates in a similar way to display the corresponding functional and sub-functional buttons associated with the Doppler state of the scanning system 10, instead of the buttons, such as Color, Doppler, Motion, and Pause associated with the Start state of the scanning system.

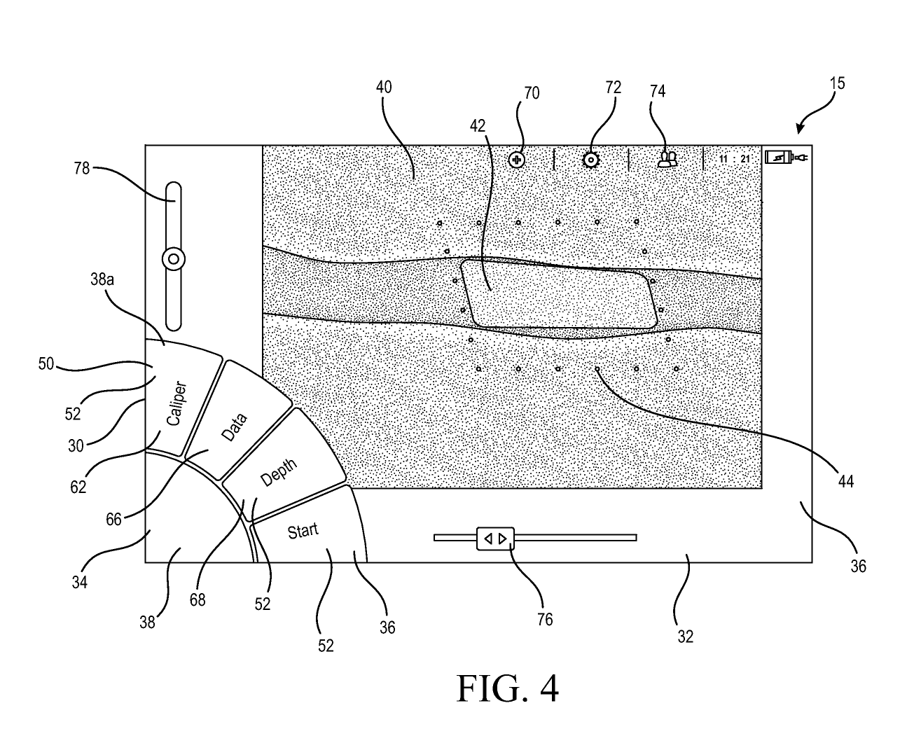

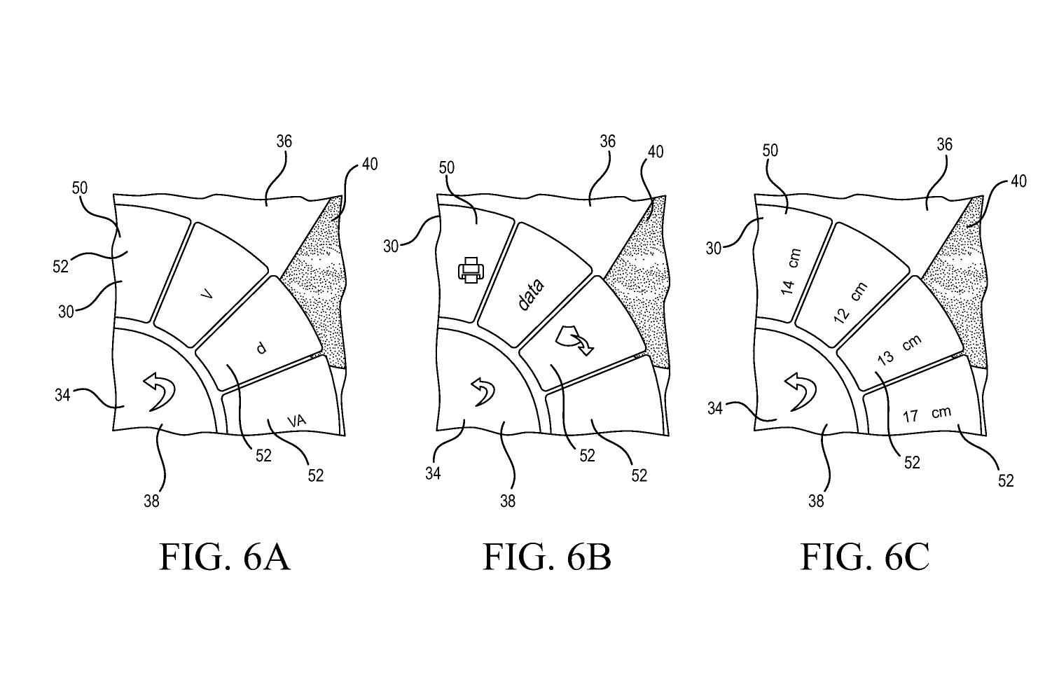

As another example, when a Pause button 60 (FIG. 7) is activated, the present system 10 transitions into the pause mode. During the pause mode, the scanned image 40 is frozen, and one or more measurements may be taken using a Caliper button 62 (FIG. 4). It is contemplated that, as illustrated in FIG. 6A, when a Measure button 64 (FIG. 5) is depressed by the finger of the operator 12 for a predetermined time period, the corresponding functional and sub-functional buttons, such as Distance, Area, and Volume buttons, are activated and displayed on the touchscreen 32.

Similarly, as illustrated in FIG. 6B, when a Data button 66 (FIG. 4) is depressed by the finger of the operator 12 for a predetermined time period, the corresponding functional and sub-functional buttons, such as Store, Print, No Steering buttons, are activated and displayed on the touchscreen 32. Also, as illustrated in FIG. 6C, when a Depth button 68 (FIG. 4) is depressed by the finger of the operator 12 for a predetermined time period, the corresponding functional and sub-functional buttons showing different values of depth for viewing are displayed (e.g., 12 centimeters, 13 centimeters, 14 centimeters, etc.). In some of these states of the scanning system 10, the virtual trackball zone 38 becomes a “return” button, to return the scanning system to the previous state.

Referring now to FIGS. 2, 4 and 7, it is preferred that, during the Pause mode, a plurality of auxiliary buttons are provided for performing ancillary functions of the present system 10. For example, in FIG. 4, a New button 70 is provided on top of the touchscreen 32 for generating a new file for the patient 42. A Settings button 72 is provided on top of the touchscreen 32 for modifying configurations of the present system 10. A Review button 74 is provided on top of the touchscreen 32 for reviewing the patient data. A window adjuster 76 is provided to adjust a location of the ROI window 44. Other auxiliary displays or icons for displaying related information, such as a date and time stamp, and a level of battery for the ultrasound electronics 15, are contemplated.

It is also contemplated that other auxiliary control tools may be displayed on the display 30 as desired. For example, in FIG. 7, a Gain slider 78 is provided to compensate a wide range of color and hue presentations of the image 40. When the trackball zone 38 of the virtual control area 36 is positioned on a bottom side of the touchscreen 32, the Gain slider 78 can be positioned horizontally above the virtual control area 36.

However, it is contemplated that the trackball zone 38 can be disposed in any corner or along any peripheral edge of the touchscreen 32 to suit different applications, and thus the orientation of the Gain slider 78 varies based on the disposition of the trackball zone. For example, if the trackball zone 38 is positioned on the left side of the touchscreen 32, the Gain slider 78 can be positioned vertically on the right side of the virtual control area 36. Other suitable arrangements of the Gain slider 78 are contemplated.

While at least one exemplary embodiment of the present invention has been shown and described, it should be understood that modifications, substitutions and alternatives may be apparent to one of ordinary skill in the art and can be made without departing from the scope of the invention described herein. This application is intended to cover any adaptations or variations of the specific embodiments discussed herein. In addition, in this application, the terms “comprise” or “comprising” do not exclude other elements or steps, and the terms “a” or “one” do not exclude a plural number. Furthermore, characteristics or steps which have been described with reference to one of the above exemplary embodiments may also be used in combination with other characteristics or steps of other exemplary embodiments described above.

What is claimed is:

1. A portable ultrasound scanning system for performing a diagnostic ultrasound scanning process of a patient, comprising:
a portable housing configured to be held in a first hand of a user of the scanning system,
ultrasound electronics of the ultrasound scanning system being contained within the hand-held housing,
a display comprising a touchscreen mounted on the hand-held housing,
a user input interface, at least a part of which is presented on the display, and being configured to receive inputs from the user via the touchscreen and to transmit those inputs to the ultrasound electronics,
a scanning probe arranged to be in communication with the ultrasound electronics and configured to be held in a second hand of the user of the scanning system,
a virtual control area being included in the user interface, the virtual control area being displayed on the touchscreen of the portable housing; and
a trackball zone being included in the virtual control area, the trackball zone performing a navigational function using a touch-sensitive manipulation method.

2. The scanning system of claim 1, wherein at least a portion of the trackball zone is displayed in one corner of the touchscreen.

3. The scanning system of claim 1, wherein at least a portion of the trackball zone is displayed along a peripheral edge of the touchscreen.

4. The scanning system of claim 1, wherein at least a portion of the trackball zone is displayed on the touchscreen in an area accessible by at least one finger of the first hand of the user that is holding the portable housing.

5. The scanning system of claim 4, wherein the entire trackball zone is accessible by at least one finger of the first and of the user that is holding the portable housing.

6. The scanning system of claim 4, wherein the at least one finger comprises the thumb of the user.

7. The scanning system of claim 1, wherein the virtual control area includes a virtual button arc disposed around a portion of the trackball zone.

8. The scanning system of claim 5, wherein the trackball zone comprises a shape having an arcuate edge portion.

9. The scanning system of claim 7, wherein the virtual button arc is accessible by the at least one finger of the first hand of the user that is holding the portable housing.

10. The scanning system of claim 7, wherein the virtual button arc has a plurality of virtual buttons displayed on the touchscreen for controlling at least one imaging system function.

11. The scanning system of claim 10, wherein the virtual buttons are each associated with different imaging system functions.

12. The scanning system of claim 10, wherein the virtual buttons are each associated with a single imaging system function.

13. The scanning system of claim 10, wherein a touching of a first of the virtual buttons causes a display of different legends on the remaining virtual buttons based on an imaging system function associated with the first of the virtual buttons.
